(19) United States
(12) Patent Application Publication
(10) Pub. No.: US 2019/0191665 A1
(43) Pub. Date: Jun. 27, 2019

(54) SYSTEM AND METHOD OF MEASURED DRUG EFFICACY USING NON-INVASIVE TESTING

(71) Applicant: Mousera, Inc, San Mateo, CA (US)

(72) Inventors: Laura Schaevitz, Los Gatos, CA (US); Daniel J. Ford, San Francisco, CA (US); Jonathan Betts-LaCroix, Belmont, CA (US)

(73) Assignee: Mousera, Inc, San Mateo, CA (US)

(21) Appl. No.: 15/851,222

(22) Filed: Dec. 21, 2017

(51) Int. Cl. A01K 1/03; A01K 11/00; A01K 29/00

(52) U.S. Cl. CPC A01K 1/031; A01K 11/001; A01K 29/005; A01K 11/005; A01K 11/006; A01K 2267/03; A61B 2503/40

(57) ABSTRACT

Systems and methods of measuring drug efficacy and side effects using non-invasive or husbandry-only testing are described. Steps include testing a cohort with a proposed husbandry-only protocol against an existing gold-standard treatment, and then validating the use of a created surrogate, non-invasive metric in place of an invasive metric. Then, the validated non-invasive surrogate metric and the husbandry-only protocols are used with an animal treatment cohort to study a new proposed treatment. A control cohort is also used, subject to the same husbandry-only testing and the surrogate metric. A statistical difference in outcomes, using one or more surrogate metrics, between the treatment cohort and the control cohort is the drug efficacy, for a drug used to treat the treatment cohort.

### FIELD OF THE INVENTION

This invention relates generally to systems and methods of automated behavioral monitoring of animals to determine health, drug efficacy, disease models, and clonal classification. More particularly embodiments relate to non-invasive testing, use of only husbandry actions and environments of the animals, and validation of behavioral models thereto.

### BACKGROUND OF THE INVENTION

Traditional drug testing in animals uses invasive testing while the animal is alive, and may require euthanizing animals. More recently, automated behavior observations and statistical processing have enabled use of large datasets to provide more consistent and revealing information. Such data is useful additionally for creating disease models and characterizing animals, such as for new clones or new treatments.

For these purposes, up to four sets of animals and associated data may be used: a positive group, often treatment of a known disease with an existing, “gold standard” treatment; a negative group, typically animals with the known disease but no treatment; a control group, using the same animals and environment as the test group with a vehicle-only or sham treatment; and a test group, animals with the known disease being treated with a drug or other treatment under test. In addition, a healthy group may be used, consisting of similar animals with no disease.

Behavioral “signatures” may be developed for each of the above groups. These signatures are statistically compared, and may also be compared against human-requested “query” data.

The prior art uses two kinds of invasive testing. The first kind uses invasive stimuli, such as air puffs, stress-inducing noise or light, or environmental changes such as temperature. The second kind uses invasive testing such as measuring tumor sizes or blood draws for blood tests. Both kinds of tests are “non-husbandry,” meaning that they are more than standard, accepted husbandry practices such as food, water, clean bedding, or circadian lighting.

Such invasive testing stresses animals, may be performed only at certain minimum intervals, and may require euthanizing the animal.

Behavioral stimuli and behavioral modeling may lack credibility in the field compared to existing, gold standards for both disease characterization and treatment.

### SUMMARY OF THE INVENTION

An embodiment uses only non-invasive testing, although disease induction or treatment may be invasive.

Another embodiment uses only accepted, standard husbandry practices for the animals.

Yet another embodiment uses a first step of monitoring behaviors using non-invasive or husbandry-only practices and correlating those behaviors with existing gold standard measurements to establish credibility, “validation,” that the new non-invasive or husbandry-only measurements are as good as, or better, than the existing gold standard. Then, a study is performed using the validated, improved, behavior monitoring and statistical processing, generating study results that may include efficacy and confidence metrics, new models, or new knowledge.

The term, “behavior,” herein, generally includes also, neurological and physiological data, unless otherwise clear from the context. The term, “behavior,” may or may not be abstract; however, embodiments observe, communicate, record, and analyze behaviors using quantified, digital data from automated, electronic sensors. Such data and such methods are not abstract.

### BRIEF DESCRIPTION OF THE DRAWINGS

FIG. 1 Two mice in a cage with sensors in a vivarium.

FIG. 2 Prior art.

FIG. 3 A block diagram showing steps of an embodiment using correlation with an existing gold standard treatment.

FIG. 4 A block diagram showing an embodiment of validating husbandry-only testing.

FIG. 5 A block diagram showing an embodiment of a method for validating and then using husbandry-only testing.

### DETAILED DESCRIPTION OF THE INVENTION

Embodiments, scenarios, examples and drawings are non-limiting.

Vivariums house a number of animals, typically test or study animals, such as mice, in a number of cages, often thousands of cages. The study animals are frequently used to test drugs, genetics, animal strains or clones, husbandry experiments, methods of treatment, procedures, diagnostics, and the like. We refer to all such uses of a vivarium as a study.

In order to study a new treatment, baseline data is required. Such data typically includes data for two sets of animals: a positive group comprising animals with a known disease treated with a known, “gold standard” treatment; and a negative group comprising animals with the known disease and no treatment. Baseline data may include a healthy group comprising similar animals without the disease. Note that a “disease” may be induced, occur spontaneously, or be built into the genetics of the animal.

During the test, typically two sets of animals are used for a study: a test group with the disease that receives the treatment under test; and a control group of similar animals that receive either a vehicle-only or sham treatment. A vehicle, for example, may be an injection comprising only saline.

Prior art uses two kinds of invasive or non-husbandry tests: first as a stimulus and second for measurement. “Invasive,” in the art, generally means inside the animal, such as injections or surgery. However, some tests and stimuli are stressful to an animal and may alter test results, such as loud sounds, unnatural lighting, inhospitable environment, air puffs, and the like. In some cases, such non-husbandry stimuli, tests or measurements may be considered “invasive,” because the alter the animals normal behavior. Husbandry refers to accepted and therefore standard practices for the care of animals, such as providing proper temperature, suitable air, a sterile (as defined in the art) environment, food, water, suitable bedding, circadian lighting, suitable exercise and nesting equipment or supplies, sufficient cage space, chewable items, and an environment free of undesirable effects on the animal, such as handling, movement to an unfamiliar cage, temperatures outside of an ideal range, unusual air, toxins or dangerous equipment, and unusual noises or activity outside the cage. Husbandry, for some studies, also includes appropriate mating, family and companionship (or lack) of other animals. For convenience, we use the term, “non-husbandry” to include tests, actions, or environment outside the scope of accepted husbandry practices, including any invasive actions on, or unnatural environment for, the animals.

Embodiments are generally free of unnecessary manual handling, manual observations, and manual rating of animal activity. Some activities, such bedding changes, placing animals originally into their home cages; removing a cage lid for replenishing water or food; or repairing dirty, moved or damaged equipment may involve manual handling, but we do not consider such activities to be non-husbandry. Such required manual activities, indeed, may be an important part of husbandry-only behavior monitoring.

We use the term, “behavior monitoring,” to include automated observing, recording, communication, and data processing.

In order to quantitatively measure the behavior of a set of animals in a study, it is necessary to have baseline behaviors. A baseline behavior set may be negative (healthy animals) or positive (animals with a known tumor). Such baseline behaviors may be known in the art, or may be measured as part of embodiments, or as part of a study.

Animals may be singly or multihoused. That is, there may be a single animal in a cage or multiple animals, typically two to five in a cage. Embodiments, claims, and drawings should be construed to include both types of housing, unless otherwise clear from the context. For multihoused animals, associating any observed behavior to a single, identified, animal in the cage is critical. See below for discussion on animal ID.

Examples of animal behavior or phenotype include but are not limited to:
performing a stereotypical “nose poke;”
use of passive exercise equipment in the cage;
interacting with another animal in a specific way;
mating, grooming, fighting or parenting;
chewing;
scratching;
burrowing and nesting, including patterns;
sniffing;
yawning;
stretching;
sleeping and sleep patterns;
normal vocalization, including ultrasonic vocalizations;
performing a physiological action or behavior related to increasing or decreasing body temperature; or the lack thereof;
selecting a preferred food or drink source over another;
sounds: type, frequency or volume; or the lack thereof;
selecting one food or drink source over another;
resisting eating or drinking;
eating or drinking;
normal or abnormal gait;
tail position;
normal or abnormal urine components;
behavioral patterns or frequency;
weight; weight gain or loss.

Non-husbandry, or invasive, tests include but are not limited to:
use of levers;
air puffs;
providing rewards for desired animal actions;
providing a penalty for undesired animal actions;
training an animal;
generating unusual sounds or light;
altering usual circadian patterns of light, temperature, air quality, etc.;
intentionally generating a startle response;
water maze;
high-tower walk test;
any specific non-husbandry stimuli to generate a desired response;
any specific non-husbandry stimuli to generate habituation of non-habituation response;
electric shocks;
bedding free cage;
electric shocks;
temperature outside of accepted husbandry bounds;
introductions of toxins or non-sterility;
unsafe equipment;
probes on or in the animal;
shaving the animal.

Husbandry activities do include:
one-time accepted animal ID marking including use of ear tags, tail tattoos, and embedded RFID;
fixed arrangement, replenishment and repair of animal furniture and husbandry supplies;
providing clean and appropriate food and water;
providing passive exercise equipment;
providing chewable, non-toxic objects;
providing space, supplies and equipment for nesting;
providing appropriate circadian lighting;
providing other animals for socialization or breeding, as appropriate for the study;
cage cleaning and bedding replacement;
adaptation to jet-lag.

Some embodiments do not use a vivarium. For example, animal studies or subjects using livestock, research animals such as monkeys or rabbits, wild animals, or pets may be performed in other environments such as on a farm or ranch, in an animal production environment, a home, a hospital, a veterinary clinic, or the wild.

It is desirable to keep vivarium animals in sterile cages. It is also desirable for sterility and for practical reasons such as cost, maintainability, and keeping foreign material out of the cage, to use a cage with no electrical penetrations.

Therefore, it is also desirable to implement sensors and testing methods that are free of electrical penetrations of the cage.

Rodents are prone to chew on almost every material in their cage. Thus, keeping sensors and electronics outside the cage is particularly important. Sensors and electronics external to cages is an important and novel aspect of some embodiments.

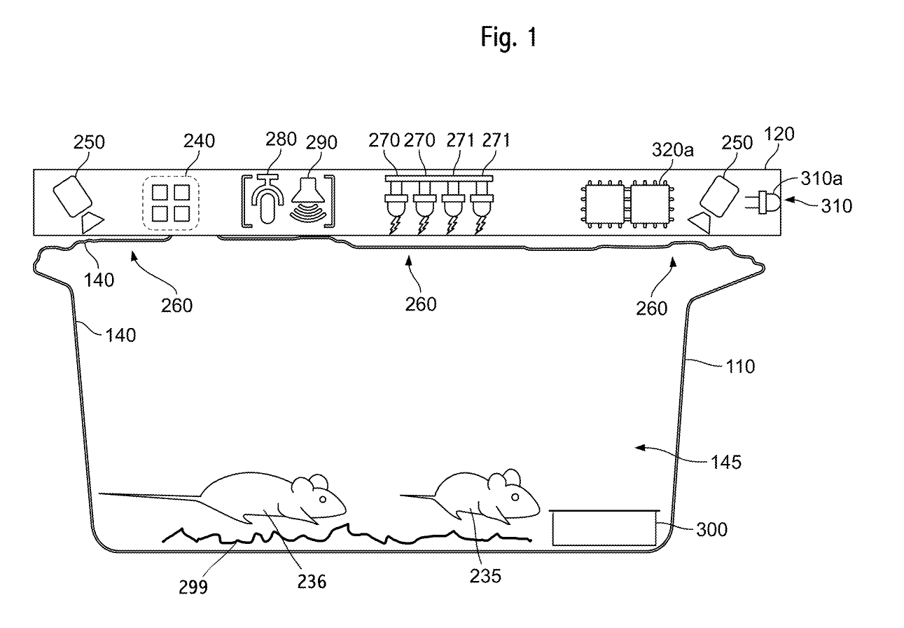

Turning now to FIG. 1, we see a schematic side view of a cage with sensors. The periphery of the cage, often constructed from clear plastic, is shown as 110 and 140, for the outside and inside surfaces respectively. The interior of the cage, ideally sterile, per the definition of sterile in this specification, is 145. Thus the sterile border is between 140 and 110. Bedding area is shown 299. A scale, typically a wireless, sterilizable, re-useable scale is shown 300. The scale may be below a water bottle, not shown, to encourage mice to climb on the scale regularly. Exercise equipment is not shown. 260 shows in three places clear areas at the top of the cage through which cameras 250 may view the inside of the cage, and through which visible light and infrared light, from LEDs 270 and 271, respectively, may enter the cage. Cages may be disposable or sterilized between studies. Ideally, and key to some embodiments, is that there are no electrical penetrations of the cage periphery, 110 and 140. Cameras, which may be still or video, monochrome, color or infrared (IR), multiple or single, are shown 250. 280 and 290 show respectively a microphone and speaker, which may be used for either ambient (vivarium) or in-the-cage audio use. 240 shows exhaust air sensors, such as temperature, humidity, ammonia concentration, and the like. 320a shows local processing electronics, which may include CPU, analog and digital processing, including video image processing, storage and communication, in any combination. 310a shows an LED pointing away from the cage, which may be used as an indicator for humans, such as the cage needs attention, or as an optical communications element. 310 shows a base, enclosure or “slab” that contains some or all of the electronics and sensors. Ideally this slab 310 is separate from the cage 110, so that cages may be easily removed, swapped, or replaced without disturbing the electronics, and similarly, all of the electronics in the slab 310 may easily be installed, serviced, updated, or swapped as slab units without disturbing the cage or its animals. Cages may slide in and out of their holding racks on rails, while the slap is mounted overhead each cage. Similarly, slabs may sit simply on supports, with electrical connection via a connector or fingers. In this way, both the electronics and the cages may be removed and replaced without disturbing the other. Other sensors may also or alternatively be used, as discussed below. Husbandry elements such as food, water and bedding are not shown. Also not shown in this Figure are supply and exhaust air ducting. These husbandry elements may also be monitored by the sensors in slab 310 or by sensors elsewhere.

Two animals are shown in FIG. 1 as 235 and 236. Here, there are two mice. As described above and below, embodiments may use a wide range of animals for studies. The identities of the two mice, 235 and 236, are distinguished in different embodiments by different sensors, such as RFID (not shown), barcodes on the animals (not shown), ear tags (not shown) or video processing via cameras 250. Identification of animals is discussed below. These two mice, 235 and 236, may be observed via the sensors and electronics to generate two distinct phenotypes, as explained below.

Microphone 280 may receive either human-range audible vocalizations or ultrasonic vocalizations, or both. This microphone may also pickup spoken information from technicians in the vivarium. Speaker 290 may be used to provide audible information to a vivarium technician, background sounds that are husbandry compatible, including white noise, or non-husbandry stimulation.

The cage hardware as shown is capable of providing some non-husbandry stimulation, such as sound and light. Stimulations of this type are not part of embodiments herein, unless otherwise clear from the context.

Either LEDs 270 and 271 may provide circadian light for the animals in the cage, or such lighting may be provided generally within the vivarium, not shown.

FIG. 1 is schematic only. Actual sensors and cage design may differ substantially from the shapes and locations shown. Embodiments may use more elements or fewer elements than shown. Husbandry elements such as a water bottle, food tray, exercise equipment, nesting locations, chewable objects, and the like are not shown. Note that this cage has no electronic penetrations.

Not all sensors are invasive, such as cameras; while others, such as probes imbedded in animals, are. Sensors such a thermometers, cameras, air monitoring, microphones, scales do not interfere with either husbandry practices are noticed by the animals are within the scope of “non-invasive” and may be within the scope of “husbandry-only,” as they may monitor husbandry practices or monitor animal health in ways that area undetectable to the animals or have no negative impact on the animal's behavior.

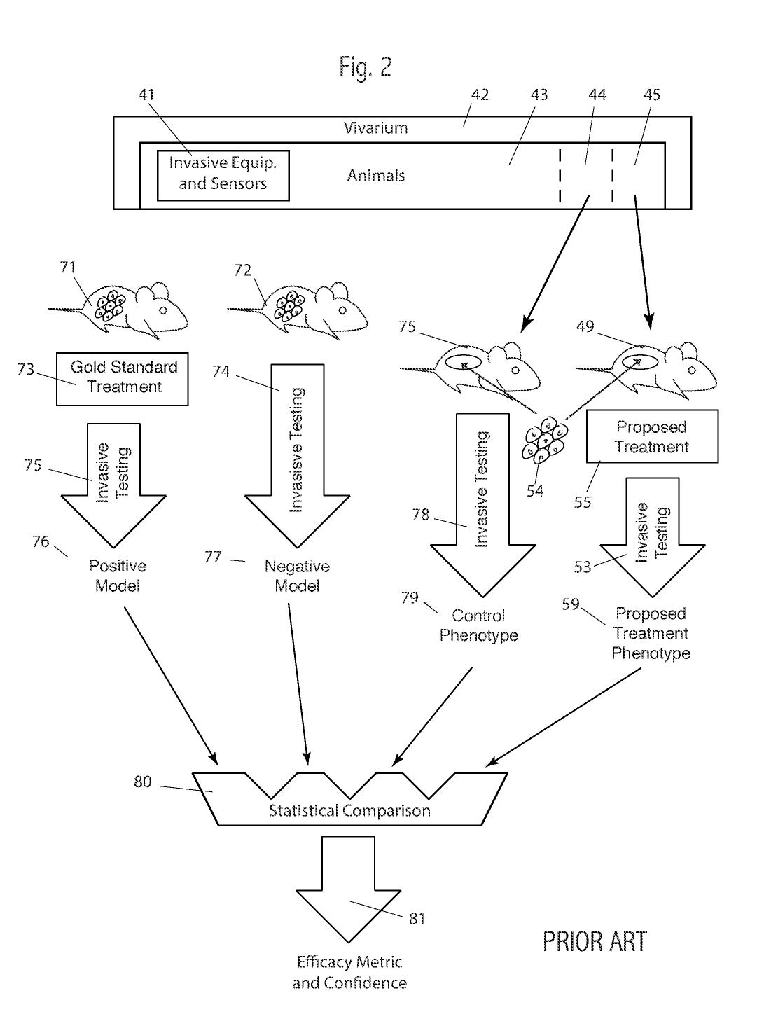

Turning now to FIG. 2, we see typical elements and protocol flow for prior art. 42 is a vivarium housing animals, 43. 41 shows invasive equipment or apparatus and sensors, typically or partially inside of one or more cages. Examples of invasive tests are described elsewhere herein, but may include air puffs or high-tower walk tests, as minimal examples. Prior art uses a mix of in-cage and out-of-cage apparatus and sensors. Typically sensors communicate data over a network to a computer system than includes statistical analysis software and may also include query input and display capabilities, not shown.

Such communications are not shown in FIG. 2. The sensors may be located in one or more cages, or external to a cage. In one embodiment the cages are free of electronic penetrations, which assists in maintaining sterile cages and in vivarium management. Sensors may be per-cage or may monitor multiple cages.

Prior art typically includes a positive and negative disease models, 76 and 77 respectively, generally available prior to a study or test of proposed treatment. Typically animals 71 and 73 for these models do not come from the same vivarium 42 as the animals used for study, 75 and 49. A positive model uses a sick animal 71 that receives a “gold standard” treatment 73, such as a known drug. After testing, typically invasive 75, a positive model 76 is created. Invasive testing is described elsewhere herein, but may include tumor measurement or blood tests, as two minimal examples. A negative model 77 is created similarly, using a sick mouse 72. However, this mouse receives no treatment. The difference between the positive model and the negative model is generally known as the efficacy of the gold standard treatment 73, and may also include side effects and confidence. Generally, a limited number of tests, such as a single test, are run on animals 71 and 72. Thus, the negative model 77 may be limited in its information or phenotype for animal 72.

Next, in the prior art, continuing with FIG. 2. we see a study being performed involving a control group comprising animals 75, from the vivarium 42 and control group cages 44, invasive testing 78 using equipment and sensors 41, generating a control phenotype, which again may be limited in information about animals 75. The study includes a group testing the proposed test treatment 55 on animals 49 from cages 45, receiving invasive testing 53, generating a proposed treatment phonotype 59. Often, the control group 75 and the group under the proposed treatment 49 are studied at the same time or at closely related times. Also the invasive testing 53 should be similar to invasive testing 75. In a basic study or comparison, the results 59 of the proposed treatment 55 are compared against the results 76 of the gold standard treatment 73. The group under test results 59 may also be compared against the control group results 79. In addition, the control group results 79 may be compared against the negative model for the disease 77. The statistical comparisons are shown generically as 80. The most common result of prior art studies is efficacy and confidence of the proposed treatment 55. In addition, side effects of treatments may be studied, compared or analyzed, not shown.

For the prior art study, a disease 54, such as cancerous cells, is introduced into animals 75 and 49, a process generally known as induction into a study.

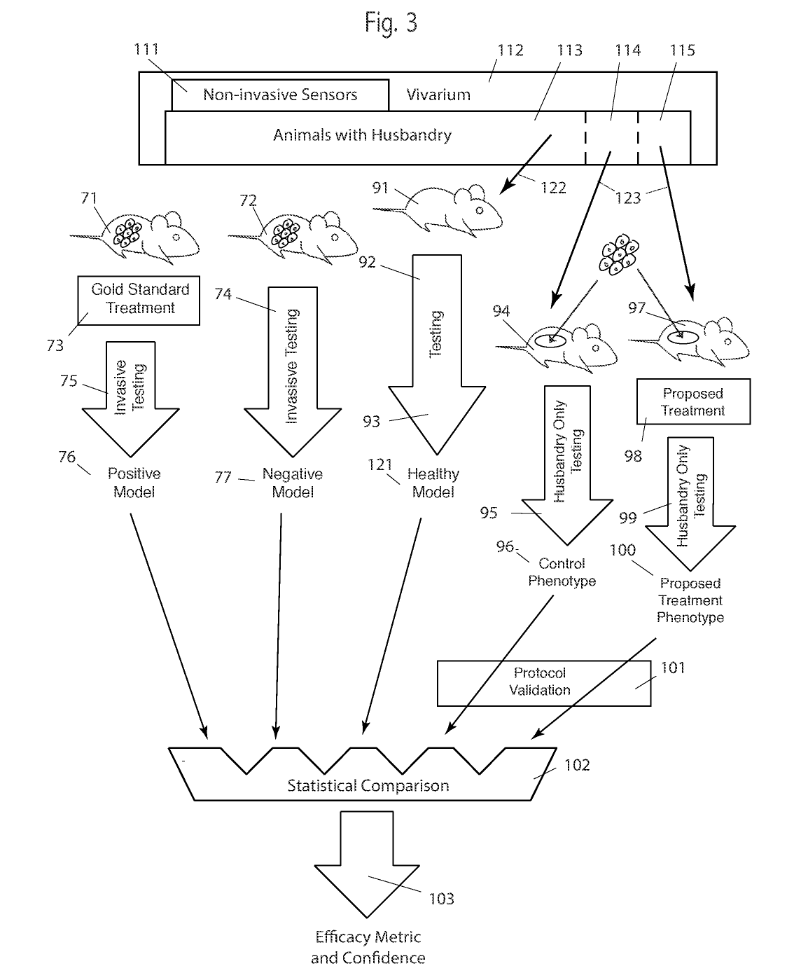

Turning now to FIG. 3 we see several, but not all, embodiments of a study of this invention. Although there are many novel elements in embodiments, the most important ones include:
non-invasive apparatus and sensors, such as 111;
apparatus and sensors external 111 to animal cages;
non-invasive or husbandry-only testing, such as 95 and 99;
validation 101 of such protocols, including validation prior to other studies using such protocol;
complex behaviors to generate phenotypes 96 and 100;
inclusion of up to five models or phenotypes for test treatment 98 efficacy 103.

Claimed embodiments include all combinations of the above.

FIG. 3 does not generally show time-based flow of events or method steps.

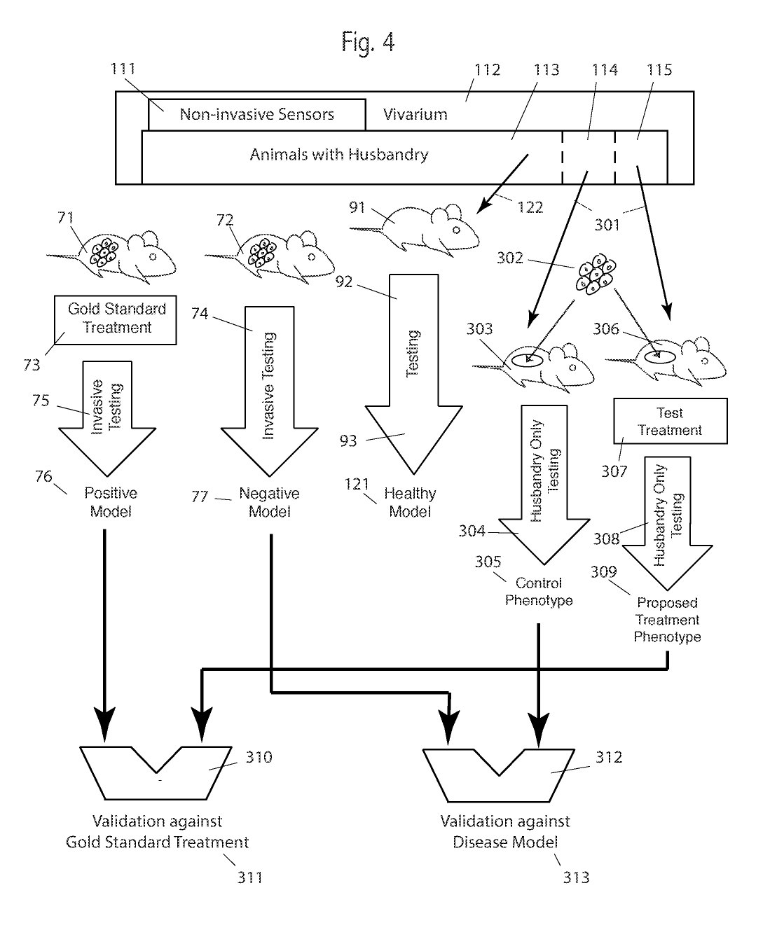

FIG. 4 provides more detail about the validation step 101 shown in FIG. 3. Unless otherwise clear from context, elements or steps identified with previously described reference designators are the same or reasonably similar for the purpose of a study.

Although numerous different comparisons may be made between various models, phenotypes, control and proposed treatment results, both existing in the field, and as results of a study, two important comparisons are shown in this Figure, without limitation. First, it is necessary to validate a non-invasive or husbandry-only test protocol 304 or 308, or both, against a gold standard treatment 73 and invasive testing protocol 75, or against a healthy model 121. Such a comparison and validation is shown 310. As a single example, invasive testing 75 may be to measure a tumor size; whereas the non-invasive testing may be to measure animal activity and breathing rate, using husbandry-only procedures 113 for the animals 306 in the vivarium 112.

Second, for some embodiments, comparison of the control group's phenotype 305 is compared 312 against a known disease, or “negative” model, 77. This comparison may be used to validate husbandry-only testing protocol 304 against the protocol used 74 to generate a negative model.

We now provide a note on the terms, “non-invasive testing” and “husbandry-only” procedures. In many cases, including construction of claims, these terms are the same or closely related, although they are not identical. Also, the terms may be used, for some contexts herein, as interchangeable. Non-invasive testing generally takes on the meaning of “invasive” from the term of art, such as any test on an animal that either penetrates the animals, such as an imbedded probe, blood tests, or euthanizing the animal; in addition, the term also include procedures outside the scope of accepted husbandry procedures, such as moving the animal outside of its home cage for a water maze, high-tower walk test, and the like. The term also includes stressing an animal outside of normal husbandry practices such as different lighting or temperature, unnecessary handling, electric shocks, air puffs, lack of bedding, rewards or punishments, and the like. “Husbandry-only” procedures or practices means required or recommend practices for the housing and well-being of the animal, which is discussed in more detail elsewhere herein.

Given appropriate equipment, sensors, data and data processing, some of which are part of novel embodiments of this invention, husbandry-only procedures are able to provide the necessary data to conduct valid studies. As a single example, monitoring normal exercise activity such as use of a running wheel or ladder may provide information related to an animal's health, motor coordination, curiosity, pain, and the like. Claim construction or alternate embodiments include embodiments where the two phrases, “husbandry-only” and “non-invasive,” are swapped.

Note that results of husbandry-only testing will, for many embodiments, include more quantity and more varied test data than prior art testing, such as 75 and 74. The results and resulting phenotypes of models, 305 and 309 may include more data and more factors to compare than positive and negative models used in the field, 76 and 77. For example, breathing rate may be recorded every minute; or weight recorded every hour, as two simple examples of more data. As another example, behaviors as surrogates for pain, motor-control, or curiosity may be provided for data such as tables or graphs showing either disease or treatment progression over time. Whereas, prior art may know only end-point conditions, particularly when a metric is only available after death. As another example, a blood draw for a blood test, for prior art protocols 75 and 74 may be performed no more frequently than weekly or daily; while surrogate behaviors may be recorded daily, hourly, or minute-by-minute.

Method steps and equipment or elements of devices or systems to validate non-invasive or husbandry-only testing protocols as surrogates for prior art, invasive testing are specifically claimed.

Continuing with FIG. 4 we see two paths of animal protocol in a study. Path 114, 301, 302, 303, 304 and 305 is for a control group. Path 115, 301, 302, 306, 307, 308 and 309 is a group for testing a proposed or test treatment 307. The control group has a correspondence with a disease model using invasive testing, path 72, 74, and 77. The group under test has a correspondence with a positive model using a gold standard treatment path 71, 73, 75 and 76.

Note that a specific difference with prior art is protocol 308 v. protocol 75. In order to establish creditably in the field that protocols such as 304 and 308 provide the same or better results than protocols 75 and 74, it is necessary to first use the same gold standard treatment 73 as the test treatment 307. Such validation means that phenotype 309 should closely resemble positive model 75. although it might comprise more temporal data and data for more individual health or disease metrics, such as pain, motor control, or quality sleeping.

Such validation typically uses an “r-squared” metric that compares the similarity of two parameters. An exact match generates an r-squared of 1.0. A poor match—no correlation—generates an r-squared of 0.0. One such comparison chart plots the prior art, accepted metric, such as tumor size, on the X-axis, with a proposed surrogate metric, such as breathing rate or exercise activity time, on the Y-axis. A high correlation, one that establishes the proposed surrogate metric, appears as a straight, diagonal line, with r-squared close to one, for the chart.

Once such validation is done—that is—a proposed surrogate metric using husbandry-only testing protocol is established and accepted, then a study may be performed where test treatment 307 is now a new drug, or other proposed but unproven treatment for disease 302.

For such a post-validation study, the proposed treatment results 309 may then be statistically compared against the control group results 305, or against a healthy model 121, or against known positive or negative models 76 and 77.

The concept of a “surrogate” metric to replace a metric requiring an invasive test is a critical element of some embodiments. For example, a prior art metric may be tumor size. A surrogate may be an animal's activity level, observed using husbandry-only procedures for the animals 303 and 306. As a second example, the prior art metric may be days until death; the surrogate metric may be breathing rate. Accurate prediction of death is valuable in the field.

Another key embodiment, and an associated benefit of the embodiment, is determining “disease response.” In a starting population of animals for study, either a control group 303 or a group under test 306, generally the starting sickness level varies. Even if every animal responds exactly the same to a treatment, such as either 307 or 73, since the starting points are different the progress and ending points will be different, too. For prior art testing, it was thus necessary to start with a large number of animals so that averaging produces meaningful results. However, with a non-invasive surrogate test, it is possible to directly measure the starting sickness of each animal individually. Unexpected results include: (1) fewer animals needed; (2) ability to measure efficacy of treatment for different starting disease levels, and (3) more accurate progression of either health or a disease; and (4) more nuanced understanding of side-effects.

Once non-invasive testing is validated, or a surrogate metric using husbandry-only metrics is validated, then, generally speaking, embodiments and use in the field moves from studies exemplified by FIG. 4 to studies exemplified by FIG. 3. Although not a perfect explanation, one may think of protocol validation 101 in FIG. 3 as all of FIG. 4. Elements in FIG. 3 are in time-sequence order. Embodiments include steps from FIG. 4 done prior to steps in FIG. 3. Generally, although not exclusively, the positive model math and the negative model path in FIGS. 3 and 4 may be the same.

Note that the healthy model, shown as path 91, 92, 93, and 121, in FIG. 3 may be either in the prior art in the field, or may be generated from the local vivarium 112 as part of embodiments. For example, prior art models and phenotypes of healthy animals 91 may not include detailed metrics or characterizations, such as can be determined by the non-invasive sensing such as 111 and husbandry-only testing such as 95 and 99. It is possible that proposed treatment 98 is better than the gold standard treatment 73. In this case the resulting phenotype 100 may not be a close match to the positive model 76. However, the resulting phenotype 100 may be much closer to a healthy animal model 121 than the positive model 76. To credibly demonstrate the superiority of the proposed treatment 98 it may be necessary to have a more comprehensive healthy model 121, and that is why it may be necessary to generate such a model using the local vivarium 112 and protocols 95 and 99. Claimed embodiments specifically include such healthy model generation, and use of that model in comparisons and efficacy results of a proposed treatment 98.

Up until now, we have mostly discussed metrics related to a disease and treatment of the disease. However, an important aspect of treatment is side-effects. These can be measured and be part of, or a parallel path, to models and phenotypes 76, 77, 96, 100, 305 and 309. For example, a proposed treatment 98 may be slightly less effective than a gold standard 73, yet generate far fewer side effects. Thus, in may be a preferred treatment. This is one reason that a healthy model 121 is desirable and part of some embodiments. Side effect measurements, characterizations, and surrogates are fully claimed as embodiments, both by themselves and as part of overall efficacy metrics and determination, including validation. Although the validation step 101 is shown for data paths for the control 96 and proposed treatment 100, it may also be used for the health model 121, too.

Yet another embodiment and associated benefit relates to the use in the prior art of indirect measurement of a disease with respect to what people really care about. For example, a blood test might measure liver damage, yet be a poor indicator of how a patient feels or functions. As another example, lung edema, as determined by weight of a lung in a dead animal, might indicate disease progress, yet a patient might not even notice moderate cases of lung edema. In this case, measuring non-invasive activities such as exercise, mobility, nesting, curiosity, grooming, and eating provides a much closer metric of how an animal is feeling than killing the animal and weighing its lungs. In such examples, the surrogate metrics are functionally superior to the prior art established and accepted disease metrics in the field. Determination or substitution, or both, of a superior non-invasive metric is a step of claimed embodiments.

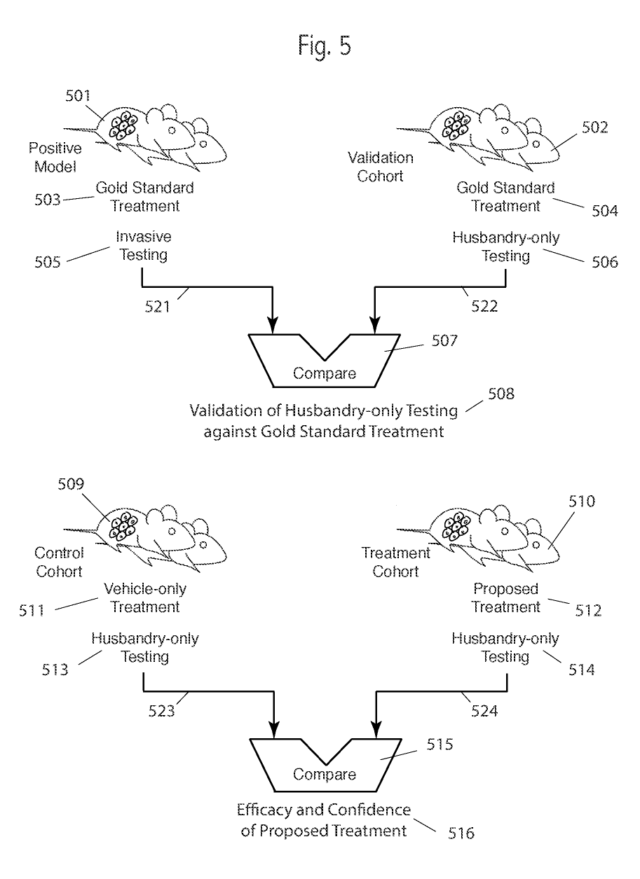

Turning now to FIG. 5, we see a more time-based flow of one embodiment. The first phase is validating a husbandry-only testing protocol; or validating a surrogate metric from the husbandry-only testing protocol, ending with 508. Steps 501 through 508 show how to generate this validation. After this validation, studies may be performed using the husbandry-only test protocol or using the surrogate metric, or both. Steps 509 through 516 show a study using this protocol or surrogate metric. Typically, multiple studies are performed, each using a different proposed treatment, after a single validation is achieved. The validation phase 501 thorough 508 may be performed repeatedly for different diseases. The compare step 508 and validation step 508 may be performed repeatedly to validate different surrogate metrics from a single disease and testing protocols.

The validation phase is broken down as follows. Two cohorts of animals 501 and 502 are diseased, often induced, but they may come by their disease in different ways. Ideally these two cohorts are as identical as possible. They receive the same treatments respectively, 503 and 504. These may be called a, “gold standard,” as this treatment is often the best available to treat the disease of 501 and 502. However, any existing treatment may be used for these steps. The two testing regiments, or protocols, are used, 505 and 506. 506 uses husbandry-only, or non-invasive testing, as described elsewhere herein. 505 uses invasive testing, typically accepted in the field as prior art, and discussed elsewhere herein.

The results of the testing of the two cohorts 501 and 502 are first, from 505, an accepted metric, requiring a least one invasive test, for measuring efficacy of the gold standard treatment. Such accepted metrics are discussed elsewhere herein and are known in the art. For example, tumor size or a blood test. From 506, a proposed surrogate metric is used, from one or more husbandry-only tests 506 or non-invasive sensors, as discussed elsewhere herein, such as breathing rate or activity.

Then in step 507 the accepted metric is compared against the proposed surrogate metric. If these are acceptably close, that is, “validated,” the proposed surrogate metric or metrics may then be used in a study, as in steps 509 through 516. “Validated” means generally accepted by one or more entities in the field, such as a researcher, a government organization, pharmaceutical company, and the like. Surrogate metrics may be “hybrids,” that is, some combination of automatically observed, communicated, recorded and quantized observed behaviors of a cohort, where the behaviors are observed using non-invasive testing, or husbandry-only testing.

A study phase following the validation phase, is broken down as follows. Two cohorts, 509 and 510, are used; here 509 is a control cohort and 510 is a cohort treated with a proposed treatment 512. The control cohort might receive a “vehicle” treatment, or a sham treatment, as known in the art. Then the same husbandry-only or non-invasive test protocol, shown in 513 and 514, is applied to the two cohorts. It is this protocol that was validated in step 508. Or a same surrogate metric is used following steps 513 and 514, where this surrogate metric was validated in step 508. The metrics from the testing of the two cohorts are compared 515, using known statistical comparisons, such as known in the art and summarized elsewhere herein. The result if this comparison 515 is the efficacy and confidence of the proposed treatment 512. Other outputs from the compare step 515 are possible, including more complex results, such as the level of side effects, for example. Ideally, one study, steps 509 through 516 produces more knowledge about the proposed treatment 514 than a single efficacy scalar and confidence.

FIG. 5 also shows four data pathways, 521 through 524. These pathways may be observed, communicated, stored in non-transitory memory, using quantified data from the non-invasive sensors, 111 in FIG. 4, in any combination. The data may be behavioral data or sensor data, or both, depending on embodiment.

Much sensor data, and further data, behaviors or phenotypes, are not instantaneous, but rather occur over time, such as the amount of movement in a 24-hour period, or the amount of food consumed over the lifetime of the animal. However, some measurements (sensor data) such as ammonia in the exhaust air, cage temperature, or time of death, are effectively measurements at one point in time. Thus, “sensor data,” “behavior,” “sets of behavior,” “derived data from sensors,” and “phenotype,” usually comprise a mix of time-interval observations, instantaneous observations, and derived observations. As non-limiting examples, a cage temperature or ammonia quantity in exhaust air are nominally single data points, where, except for correction (e.g., offsets, linearization, or calibration), come directly from sensors. Other data, such as sleeping patterns, eating patterns, activity patterns, or exercise quantity, are readily derived from a sequence of sensor data. Some data, such as time of death, or detecting death itself, required some processing of sensor data. Nonetheless, all such direct, time-interval, or derived data from sensors is, “sensor data,” in some embodiments.

In another embodiment, data 521 may be a known disease or health metric and data 522 is a proposed surrogate metric. Step 508 is validation of the surrogate metric in place of the known metric. Data 523 and 524 comprise the proposed, now validated, surrogate metric from non-invasive testing 513 and 514. Embodiments substitute “non-invasive” testing with “husbandry-only” testing, and vice versa. Embodiments substitute “validation of husbandry-only testing” with “validation of a surrogate metric,” and vice versa. “Invasive testing” may include a non-invasive element.

FIG. 5 shows two paths, that is, two comparisons. First, path 501, 503, 505, 521 into 507 is versus the second path 502, 504, 506, 533 into 507. This comparison may be considered a comparison between: (a) a disease plus gold standard treatment plus invasive testing v. the disease plus gold standard treatment plus husbandry-only testing. Other comparison scenarios, included in any combination as embodiments, include (b) the disease plus a sham or vehicle-only treatment plus invasive testing v. the disease plus a sham or vehicle-only treatment plus husbandry-only testing; and (c) no disease (e.g., healthy) plus a sham or vehicle-only treatment plus invasive testing v. no disease (e.g., healthy) plus a sham or vehicle-only treatment plus husbandry-only testing. Correlations of metrics for any combination of the scenarios (a), (b) and (c) may be used, and are claimed embodiments. Ideally, all three comparisons would yield a high correlation of one or more prior art invasive metrics and the non-invasive metric. Scenario (a) may be viewed as a positive model; scenario (b) may be viewed as a disease model; and scenario (c) as a healthy model.

Not shown in FIG. 5, but discussed elsewhere herein, is the use of healthy animal models or an existing negative model. These models may be substituted, for example, for path 502 through 506, or path 509 through 513, or both, to first validate a husbandry-only testing protocol or a surrogate metric, and then use such validated testing protocols or surrogate metrics for use in a study, steps 509 through 516. Although the comparison steps 507 and 516 are shown with only two inputs, more inputs to statistical comparison methods may be used, such as in step 102 in FIG. 3.

The cohorts shown in FIG. 5: 501, 502, 509 and 510 may be singly-housed or multi-housed animals.

Specifically claimed are systems that implement the claimed, described or shown embodiments or claims, using hardware described, shown or claimed, or hardware described, shown or claimed adapted to perform any single or combination of method steps. As non-limiting examples, such a claimed system may include a vivarium with animals, a system for performing validation of a proposed surrogate metric, or a system for performing a study using a validated surrogate metric.

Specifically claimed are devices adapted to implement the claimed, described or shown embodiments, claims or method steps.

Claimed embodiments include placing or using non-invasive sensors wholly outside an animal cage, with the exception of a wireless weight scale inside the cage, wherein the animal cages are free of electronic penetrations.

Claimed embodiments include placing or using animal identification devices on each animal in a multihoused cage. Claimed embodiments include recognizing such animal identification devices using machine vision in infrared light. Such placing or using of animal identification devices may be invasive to the animals, notwithstanding the use of non-invasive or husbandry-only testing on the animals.

Claimed embodiments include methods, systems and devices using sensors packaged in a sensor block that is free of permanent connection to a cage, such that either a cage or a sensor block may be replaced or moved within a vivarium without the use of tools. Claimed embodiments include at least one sensor in the sensor block adapted to read a cage ID. Claimed embodiments include the at least one sensor in the sensor block is a video camera that is the only video camera in the sensor block. Claimed embodiments include methods and systems wherein the combination of an animal ID to uniquely identify animals within a multihoused cage with a cage ID for the multihoused cage uniquely identifies any animal in one study.

Claimed embodiments include a validation step, of either a test procedure or a surrogate metric, comprising an r-squared correlation between a known invasive test metric and a proposed surrogate metric from a husbandry-only or a non-invasive test procedure. Suitable r-square values for validation may be in the range of 0.3 to 1.0, 0.5 to 1.0, 0.7 to 1.0, 0.8 to 1.0, 0.9 to 1.0 or 0.95 to 1.0.

Claimed embodiments include methods, systems and devices measuring efficacy, validating or studying non-drug treatments. Claimed embodiments include methods, systems and devices to use or validate surrogate metrics using non-invasive testing. Claimed embodiments include methods, systems and devices that use or are husbandry-only testing procedures.

Animals may include rodents such as mice, rats or guinea pigs; or rabbits, or livestock, or research animals, or pet animals, or even humans. In some embodiments, alternative and appropriate housing for such animals, such as a barn, farm, veterinary clinic, home or hospital may be used in place of a vivarium. In some cases the animals may be wild. Sensors are adapted to detect, observe, and communicate the behavior, physiology parameters, husbandry metrics, animal ID, and environmental conditions. Storage, analysis, and communication of such information may be included in the sensors or separate. Suitable sensors measure cage, air and animal temperature, air and cage humidity, environmental light, animal weight, animal ID, such as barcodes or RFID, animal activity, including motion, exploration, parenting, fighting, nose pokes, exercise wheels, eating, drinking, urinating, defecating, cleaning themselves or other animals, burrowing, animal sounds and noises, ammonia in the cage or exhaust air, and CO2 concentration. Sensors may include cameras, including still and video, color or monochrome, visible or infrared light. Some embodiments use auxiliary infrared lighting, or other light spectra to which the animals are not sensitive or are less sensitive. Some embodiments may use intermediate sensors or indicators, such as pH detecting chemicals in the bedding, or a wireless scale or exercise wheel. Husbandry parameters may be measured, such as water, food, condition of bedding, and exercise. Social behaviors including fighting, mating and parenting may be observed and measured. Image analysis is often used to detect, differentiate, identify, quantify, store, compare and communicate the above or other behaviors or characteristics. The term behavior is typically broad, including internal and external behaviors and physiological parameters, such are urine and breath components, unless specific narrowness of a behavior is stated, specifically claimed or indicated. All lists in this paragraph are non-exhaustive and non-limiting examples.

The use of animal studies, such as mice in vivariums, is a critical step in the testing and approval of new drugs and other treatments for cancer and other diseases.

Many cancers types, in particular, glioblastoma, are characterized by morphology of a lump of cancerous or neoplastic cells. Most generally, the cancer starts out as an isolated mass of non-normal cells, then grows exponentially to a larger mass, and then the larger tumor mass interferes mechanically with the function of the organ or location in the body where it is located. Finally, it metastasizes locally and then throughout the body. Cancer growth is often measured and quantified by the size, such as the diameter, of the tumor mass. Cancer treatment may be quantified by the rate of growth of the tumor mass.

Prior art typically measures the size of the tumor mass directly, such a visual, mechanical, manual measurement using calipers. Manual, semi-automated, or automated size imaging technologies such as fluorescence, phosphorescence, bioluminescence, light emission, radiation emission, and similar direct measurements of mass volume, diameter, or image area may be used. Such measurements may be taken of a tumor growing in a live animal, or of a removed tumor, or of a tumor in an animal that has died. However, all such measurements are invasive.

Such prior art techniques of invasive tumor mass measurement suffer from a number of weaknesses in addition to be being invasive, including inconsistent measurement, difficulty and cost of measurement, and the cost and speed of manual measurement. For some tumor locations, these problems are particularly acute. Due in part to these weaknesses of the prior art, the scope of studies, the timeliness of studies, and the repeatability of studies is often less than desired.

When animals are multihoused, that is, more than one animal in a cage, it is critical that the unique ID of each animal be associated with observed behaviors. This is not a trivial problem, particularly in an automated environment and particularly in one using monoclonal animals that may appear virtually identical. Therefore, we discuss automated identification methods and such methods are important and novel in some embodiments.

Various methods of identifying an animal are used in different embodiments. One method comprises short-distance RFID, which may use animal ear RFID tags or embedded RFID tags and RFID sensors outside the cage. Another method comprises using video for identification, which may use animal size, coloration, unique natural or artificial body elements, such as body modifications or affixed tags, for example, to provide or to assist in the identification. Another method comprises use of an animal scale: animals of distinct weights may be identified when that animal is on the scale. Yet another method uses bar codes or other artificial markings, which may be tattooed on the animal's tail or other location. Such bar codes may be read via cameras and bar code recognition software. Yet another method uses ear notches, which may be read via cameras and image recognition software.

Another method of identifying an animal is to combine technologies. For example, an animal may be first identified using an RFID when the animal is within a small RFID range, and then tracking the movement of that animal using video tracking software. Yet another method is by exclusion: if all of the other animals in a cage are identified, then the one remaining animal is also identified.

Yet another method to identify animals is by the sounds they make.

Yet another method to identify animals is by observing behavior unique to that animal.

Various methods are used in various embodiments to detect the location of an animal in a cage. One method uses short-range RFID. For example, RFID sensors may be placed at one or more locations around the perimeter of a cage, such as at the corners, of the center of the sides, and the like. When an animal comes within range of a sensor its location is then known.

Another method of detecting the location of an animal is by activity on a device, such as an exercise wheel, or on a scale. Such a device may be fully wireless, such that animal weight data or exercise data may be collected automatically, continuously or continually, without any human, manual input. In some embodiment the exercise wheel is disposable. In some embodiment the scale is sealed such that its exterior may be sterilized between studies, and the scale re-used. In some embodiments the scale is free of components physically accessible to the animals in the cages that can be chewed by the animals in the cages.

Yet another method of detecting the location of an animal is the use of an animal sensor outside of the cage, with a directional range or a short range. Examples of such detectors include thermal detectors, capacitive sensors, and motion sensors.

In some embodiments, the identification and location of an animal may be combined using the same sensor or technology, or by using overlapping elements of sensors. For example, a single RFID sensor may be used to both identify an animal and know that it is within range of the sensor. As another example, a single video signal from a single camera may go to two separate image processing elements, one for animal identification and one for animal location.

In some embodiments, real-time animal tracking within the cage may be used as part of both identification and location. For example, if an animal has a known ID and a known location, by tracking the location of the animal within the cage the ID remains known and the tracking algorithm updates the location.

Sensors, such as shown in FIG. 1, communicate directly or indirectly, via a network or other connections, wired, wireless, optical or audio, using digital or analog encoding, to a computer system, which may be local or remote, monolithic or distributed. Such communications are known in the art and not shown in Figures. The sensors may be located in one or more cages, or external to a cage. In one embodiment the cages are free of electronic penetrations, which assists in maintaining sterile cages and in vivarium management. Sensors may be per-cage or may monitor multiple cages.

### Statistics and Data Comparison

We now discuss comparison steps. In general, statistical analysis is used. However, some analysis use numerical analysis is not universally regarded as the domain of statistics. The core tools of statistical analysis for data sets like those of the relevant embodiment are well known in the art, although some embodiments incorporate novel variations, implementations, improvements and applications. The software suite known as MATLAB®, from The MathWorks, Inc., Natick Mass., provides a well-known, extensive set of tools that may be configured and used in a wide array of combinations. There is no requirement to use any of these commercially available tools. This URL, as of the date of this document provides lists of both commercial and open source statistical software:

https://en.wikipedia.org/wiki/List_of_statistical_packages.

Software tools and methodology for phenotype comparison, in element or step 60 include a non-limiting list of:
principal component analysis,
principal component regression,
linear regression,
time series analysis,
Markov analysis and models, and
clustering.

Other well-known steps that may be used to reduce and improve raw data, typically prior to statistical analysis include the non-limiting list of:
smoothing,
averaging,
outlier elimination,
slope determinations, such as least-squares-fit,
known curve determinations (e.g., exponential growths), such as least-squares-fit, and
decimation.

Clustering is particularly useful when analyzing large amounts of data in multiple dimensions. For example, the many known indicators of side effects may be clustered to identify common combinations. Then, treatments may be compared to find the nearest cluster to the particular combination of side effect behaviors observed from the treatment. Clustering algorithms are also good at creating a single metric, a “distance” in such a multi-dimensional space. Such a single metric is a useful summary or first-level characterization of a treatment or classification. Such a single metric may be a surrogate metric, such as might be validated. Please refer to discussion on surrogate metrics elsewhere herein. Quantified behaviors may be in a multidimensional space, as evidenced by both the list of possible attributes measurable by sensors the list of behaviors. Each sensor's output and each namable behavior may be considered as one dimension, and time-related variations (such as activity level during the day compared to activity level at night) considered as additional dimensions. A clustering algorithm then determines the “distance” in this multidimensional space. These distances may then be used as “single dimensional” metrics, as described elsewhere herein. The clustering algorithm could also define a “scale” for this distance. Such distances may be normalized to have a value between zero and 100, inclusive.

Other statistical and numerical methods also produce results similar to the “distances” discussed in the above paragraph, and these distances may be used similarly.

### ID Tag Image Recognition

Additional statistical methods starting with image-based data may be used to identify, compute, compare and store sensor data. Typically, a “classifier” is top-level method, which then uses “features” within the classifier. Classifiers include: SVM, cascade classifier, boosted forest, random forest, and ANN (Artificial Neural Networks for a large class). Features include ORB, SIFT, SURF, HOG, Haar-like features, and Viola-Jones. Either features or raw pixels may be analyzed using CNN (Convolution Neural Networks), R-CNN, or YOLO classifier, typically within a small area of a large image, such as a video frame. Additional information about these methods may be found in the list below:
https://en.wikipedia.org/wiki/Haar-like_feature
https://en.wikipedia.org/wiki/Cascading_classifiers
https://docs.opencv.org/3.3.0/d7/d8b/tutorial_py_face_detection.html
https://www.tnt.uni-hannover.de/papers/data/977/scia2013_baumann.pdf
https://www.learnopencv.com/image-recognition-and-object-detection-part1/
https://en.wikipedia.org/wiki/Artificial_neural_network

The above references were retrieved on 11 Dec. 2017.

Electronic observation, isolation, classification, quantification, analysis, communication and display of animal behaviors are critical steps in methods of embodiments as are the systems and devices that are used to perform such steps. We may generally divide video-base data analysis into the following four groups:
(a) Video image recognition to extract data that feeds the next step(s), such as animal location in a cage, animal identification, animal activity, biological indicators, etc.
(b) Extracting quantitative behaviors from the above, such as sleeping/awake/eating cycles, time and quantity of movement, abnormal behavior such as a limp, tremor or fighting, patterns of normal behavior, such as burrowing, exploring, mating, nurturing and exercise.
(c) Comparing data from the prior step(s) to baselines behaviors to provide some observable and meaningful, quantitative comparison. Baselines may be negative (healthy animals) or positive (animals with known tumors).
(d) Displaying behavior differences in the form of graphs and other visual forms. This includes any final summary, such as numerical treatment effectiveness within a statistical probability.

Each of the above data analysis and presentation may be well known methods, and are outside of claimed embodiments. However, one or more novel methods may be used in one or more of the above steps and are claimed in the scope of one or more embodiments. Included in the claimed scope are graphs showing multiple behaviors, individual and combined metrics, of the various different phenotypes discussed herein, on timelines. Although we use the terms “video” and “image recognition,” these are exemplary only, with no exclusion of other methods of acquiring data, such a RFID based motion sensing, exercise wheel activity sensing, one or more weight sensors, one or more motion sensors, thermal sensors, embedded sensors, solid state chemistry, molecular and cellular sensors, and the like.

For step (c) above, known analysis methods include multivariate analysis and clustering analysis.

### Definitions

Behavior and behaviors—see text above, including phenotype. The words, “behavior” or “behaviors,” may substitute, in claims, specifications and drawings, for some embodiments, for “sensor data.” The phrase, “behaviors derived from sensor data” or “behaviors responsive to statistical analysis of sensor data,” may substitute, in claims, specifications and drawings, in some embodiments, for “sensor data,” where statistical analysis may comprise any methods described herein.

Communication—may be electromagnetic, optical or audio. Audio comprises sub-audio and ultrasonic audio.

Computer—may be local, distributed, in the cloud, mobile, or any combination. May be one or more computers, or a system of computers.

Continuous collection of data—continuous means repeated substantially without unnecessary gaps in collection time intervals, subject to the inherent limitations of the sensors, communications and data recording capability of the system or method; and the nature of the data collected. This “continuous” may be compared against manual data observation which might be performed hourly or daily, for example, but which could be observed more frequently if sufficient personnel were available to perform the observations. Such continuous collection of data may, in some embodiments, also occur during environmental times where manual observation is difficult, such as in darkness.

Electromagnetic radiation—may be visible or IR light, for example, imaged by a still or video camera. May be digital or analog radio signals, such as used by RFID, Bluetooth, WiFi, or other standard or proprietary communications. May be analog or digital optical communications.

Ear tags—a form of mechanical, visual identification of animals that are multihoused. Other forms of visual identification such as tail tattoos, or ear notches, or visible tags place on animals in locations other ears, may be substituted.

IR LED—any LED that is capable without limitation, by its radiation, of causing an animal within its directed radiation to increase in body temperature, that is, skin temperature or internal temperature, by an amount detectable by the animal, as observable. Note that the spectrum of the IR LED may or not be predominantly in the infrared with respect to the visible spectrum. IR LEDs may be used to increase sensitivity of video or still image cameras, or to increase contrast or other sensitivity to animal fur or skin. Note that “thermal” cameras are normally sensitive to spectra at much longer wavelengths that traditional “IR.” However, in some cases, the term IR may be used to indicate thermal imaging.

Normal living temperature—a temperature range suitable for an animal to live normally or a temperature range appropriate for specific animal study. This may be Ta plus or minus a predetermined range, or an industry accepted range for use of the applicable laboratory animals in the applicable study.

Pathogen-free—means the population of microbes, including but not limited to bacteria, viruses, prions and toxins, relevant to the experiment, are sufficiently reduced to meet the needs of the study, or to not impact the health, performance or behavior of the target animal population or of the workers.

Primary cage—the cage in which an animal spends more time than any other cage. Of note, there is a related term of art: “home cage.” The definition of primary cage is, in some embodiments, the home cage. An aspect of home cage/primary cage deals with the fungibility of the actual cage itself. Each time a cage is changed, the physical cage is generally either disposed or removed for washing, and replaced by a clean cage. The new physical cage is considered the same primary cage. A primary cage may sometimes be distinguished from a non-primary cage by the purpose of the cage. For example, a home cage may be for living in, as compared to an experimental cage to which the animal is transferred that is equipped or located for one or more particular experiments for the applicable study.

Quantity of tumor cells—any measured or measurable quantity of a source tumor or related tissue, cells, or tumor-related chemicals, such as a carcinogen. Such quantities or counts may be computed or inferred.

Regimen or protocol—is defined broadly to include any combination of treatments. A regimen may match one of the treatments, after adjusting for differences between the test subjects and the patient(s). However, one or more selected regimen may include combinations of treatments not tested directly, or different doses or different routes or different timing, or the use of similar drugs to those tested. A regiment may include treatment elements not tested in the study. What is important in selecting a regimen is that the selection is responsive to the phenotypes and differences between the phenotypes; that is, responsive to the steps in the method. The steps of the claimed methods or the use of claimed devices or systems informed the selection of a regimen.

Sealed enclosure—an enclosure that limits against entrance or exit of pathogens that impact or alter study results, or alter the credibility or repeatability of study results. The enclosure may not be sealed in the hermetic sense.

Sensor—may or may not include the use of local or remote processors, and may or may not include local or remote software executing on the local or remote processors. Sensors may or may not communicate over a network. Multiple sensors may or may not include common elements.

Set, subset or group—one or more, unless stated otherwise. A subset may include the entire set of which it is a subset, unless stated otherwise. When a first subset and a second (or third) subset are identified, these subsets are assumed to not be identical, although they may overlap, unless stated otherwise. In some embodiments, the different subsets have no overlapped members.

Sterile—pathogen-free for the purposes of the study. The exact level of sterility and the exact pathogens depends on the study and animals used. In some cases, sterile means, “free of undesirable pathogens.”

The primary cage is different from special purpose, behavioral-measurement, behavioral-detection, or behavioral-observation cages that are generally used for only a short time for the duration of a particular test due to cost and mindset.

Treatment drug—may in some contexts be a control, such as saline. Drugs may be administered via multiple routes. That is, treatment may also be “no treatment,” “benign treatment,” or “vehicle” treatment, such as might be used to establish a baseline, positive, or negative control group, data or sample.

Treatment—a treatment may be administration of a drug, but construction of this term is broader to include any action or set of actions that could reasonably, in the art, constitute a treatment for a disease or condition.

Visible light—Free of visible light means the ambient light is sufficiently low and in a spectrum such that the animal's physiological state and behavior are consistent with its natural physiological state and behavior at night.

Xenograft—used herein to mean its medical definition, roughly tissue outside of its normal or original location or species of origin. It is not necessary, in the definition we use, that the xenograft is from another species. The xenograft could be a tissue sample where the source, (e.g., a patient) is a different animal that the one receiving the xenograft. Also note that in many cases the tissue source of sample is “amplified” before use. A common method of amplification is growth in vivo or in vitro. This amplification may happen multiple times before the tissue sample (our “xenograft”) is used in studies for embodiments herein.

May, Could, Option, Mode, Alternative and Feature—Use of the words, “may,” “could,” “option,” “optional,” “mode,” “alternative,” “typical,” “ideal,” and “feature,” when used in the context of describing this invention, refer specifically to various embodiments of this invention. Described benefits refer only to those embodiments that provide that benefit. All descriptions herein are non-limiting, as one trained in the art appreciates.

The term, “behavior,” may or may not be abstract; however, embodiments observe, communicate, record, and analyze behaviors using quantified, digital data from automated, electronic sensors. Such data and such methods are not abstract. For all embodiments, claims, drawings, examples, scenarios, the term or use of “behavior” or “behaviors” may be replaced by “digital, quantified data from electronic sensors proximal to cages comprising animals,” where such quantified data may or may not be directly associated with known, named behaviors, where such substitution provides no change to scope.

All examples are sample embodiments. In particular, the phrase “invention” should be interpreted under all conditions to mean, “an embodiment of this invention.” Examples, scenarios, and drawings are non-limiting. The only limitations of this invention are in the claims.

Ideal, Ideally, Optimum and Preferred—Use of the words, “ideal,” “ideally,” “optimum,” “optimum,” “should” and “preferred,” when used in the context of describing this invention, refer specifically a best mode for one or more embodiments for one or more applications of this invention. Such best modes are non-limiting, and may not be the best mode for all embodiments, applications, or implementation technologies, as one trained in the art will appreciate.

All numerical ranges in the specification are non-limiting examples only. Use of curly braces in claims indicates a Markov set.

Embodiments of this invention explicitly include all combinations and sub-combinations of all features, elements and limitation of all claims. Embodiments of this invention explicitly include all combinations and sub-combinations of all features, elements, examples, embodiments, tables, values, ranges, and drawings in the specification and drawings. Embodiments of this invention explicitly include devices and systems to implement any combination of all methods described in the claims, specification and drawings. Embodiments of the methods of invention explicitly include all combinations of dependent method claim steps, in any functional order. Embodiments of the methods of invention explicitly include, when referencing any device claim, a substitution thereof to any and all other device claims, including all combinations of elements in device claims.

We claim:

1. A method of measuring efficacy of a new therapeutic treatment for the treatment of a first disease comprising the steps:
placing in a first vivarium a first cohort of animals [502] comprising the first disease, the “validation cohort,” in one or more cages;
placing in a second vivarium a second cohort of animals [501] comprising the first disease, the “positive model cohort,” in one or more cages;
wherein the first and second vivariums may be the same vivarium;
treating both cohorts with a gold standard treatment [503, 504];
performing a non-invasive testing [506] on the validation cohort, wherein the non-invasive testing is free of invasive tests on the animals;
performing known invasive testing [505] on the positive model cohort;
collecting, communicating. and recording, automatically, sensor data [521, 522] of both cohorts;
comparing statistically [507] the sensor data from both cohorts;
validating [508] the non-invasive testing as suitable for testing the new therapeutic treatment of animals with the first disease in place of the gold-standard procedure;
treating a treatment cohort [510] of animals and a control cohort [509] of animals, both with the first disease, wherein the treatment cohort but not the control cohort receive the new therapeutic treatment [512], and then both cohorts receive the non-invasive testing [514, 513]; and
collecting, communicating, and recording, automatically, sensor data [524, 523] of both cohorts [510, 509];
wherein the efficacy [516] of the new therapeutic treatment is a statistical difference between the sensor data [524] of the treatment cohort versus the sensor data [513] of the control cohort.

2. A method of measuring efficacy of a proposed therapeutic treatment for the treatment of a first disease comprising the steps:
placing in a first vivarium [112] a first cohort of animals [509] comprising the first disease, the “control cohort,” in one or more cages;
placing in a second vivarium a second cohort of animals [510] comprising the first disease, the “treatment cohort,” in one or more cages; wherein the one or more cages of both cohorts are equipped with non-invasive sensors [111] outside of each cage [114, 115];
wherein the first and second vivariums may be the same vivarium;
performing the proposed therapeutic treatment [512] on the treatment cohort;
performing a control treatment [511], free of the proposed therapeutic treatment, on the control cohort [509];
collecting automatically, using the non-invasive sensors, sensor data [523, 524] from both cohorts;
communicating and recording the sensor data [523, 514] automatically, from both cohorts; and
comparing statistically [515] the sensor data of the control cohort [509] with the treatment cohort [510];
wherein the measured efficacy [516] comprises a difference scalar and a confidence scalar responsive to the comparing.

3. A method of validating of a surrogate metric for use in an animal study comprising the steps:
placing in a first vivarium a first cohort of animals [502] comprising the first disease, the “validation cohort,” in one or more cages;
placing in a second vivarium a second cohort of animals [501] comprising the first disease, the “positive model cohort,” in one or more cages;
wherein the first and second vivariums may be the same vivarium;
treating both cohorts with a gold standard treatment [503, 504];
performing a non-invasive testing [506] on the validation cohort, wherein the non-invasive testing is free of invasive tests on the animals;
performing known invasive testing [505] on the positive model cohort;
recording a known disease metric [521] from the invasive testing on the positive model cohort;
collecting, communicating and recording, automatically, sensor data [522] from the non-invasive testing on the validation cohort;
computing a surrogate metric responsive to the sensor data;
comparing statistically [507] the known disease metric with the surrogate metric; and
validating [508] the surrogate metric when a statistical match between surrogate metric and the known disease metric is higher than a predetermined validation threshold.
